(19) United States
(12) Patent Application Publication
(10) Pub. No.: US 2024/0410354 A1
(43) Pub. Date: Dec. 12, 2024

(54) PERISTALTIC PUMP

(71) Applicant: B. Braun Avitum AG, Melsungen (DE)

(72) Inventors: Marco Jakobi, Uder (DE); Oliver Schaefer, Neuenstein (DE); Bruno Stenzel, Hann. Muenden (DE)

(21) Appl. No.: 18/735,662

(22) Filed: Jun. 6, 2024

(30) Foreign Application Priority Data
Jun. 7, 2023 (DE) 10 2023 115 077.4

(51) Int. Cl. F04B 43/12; A61M 1/36

(52) U.S. Cl. CPC F04B 43/1276; A61M 1/36225; A61M 2205/103

(57) ABSTRACT

A peristaltic pump for conveying fluid in an extracorporeal blood treatment device includes a housing having a rotor with a rotor axis and a squeeze element in the pump bed. The housing also has a support surface extending arc-shaped about the rotor axis and spaced radially from the rotor. The support surface is arranged to support a tube segment insertable between the support surface and the rotor. The rotor has a swing arm for holding the squeeze element, which is pivotably movable and spring-mounted on the rotor, an insertion aid, with which the tube segment can be pressed into the pump bed during a threading process, and tube guiding elements, with which the tube segment is centrally aligned with the squeeze element. The insertion aid and tube guiding elements are integrated into the swing arm adjacent to the squeeze element in a rotational direction of the rotor.

### CROSS-REFERENCE TO RELATED APPLICATION

This application claims priority under 35 U.S.C. § 119 to German Application No. 10 2023 115 077.4, filed on Jun. 7, 2023, the content of which is incorporated by reference herein in its entirety.

### FIELD

The present disclosure relates to a tube roller or peristaltic pump, resp., i.e. a positive displacement pump, in which the medium to be conveyed is forced through a tube by external mechanical deformation of the tube.

### BACKGROUND

Tube roller or peristaltic pumps are frequently used for conveying fluid, in particular blood, in a device for extracorporeal blood treatment, in particular in a dialysis machine. The fluid is conveyed via the peristaltic pump from a low-pressure side to a high-pressure side, wherein an elastically deformable fluid line arranged between the low-pressure side and the high-pressure side in the form of a tube segment, which is referred to as a pump segment, is deformed, in particular squeezed, between a support surface of a pump bed and a rotor with at least two squeeze elements rotating relative thereto.

A peristaltic pump, as known, for example, from document EP 1 749 549 B1, features two squeeze elements 320 diametrically offset from each other in the form of spring-mounted pinch rollers are provided on/in the rotor 310 rotatably mounted in the pump housing 305 with the pump bed 307—as shown schematically in FIGS. 8 and 9—and the support surface 330 is formed by a circular segment area which extends over a sufficiently large centering angle greater than 180°. The pinch rollers are rotatably mounted on rockers or swing arms 341, which are pivotably movably fixed to the rotor 310 via a hinge pin 338 with axis A338 and are acted upon by a pressure spring 336 with a force FR directed radially outward (see FIG. 8).

In order to put the peristaltic pump into operation, it is necessary to guide a tube segment not shown in detail in FIG. 8, i.e. the pump segment, from a low-pressure-side terminal section PN arc-shaped above the rotor 310 following the support surface 330 to a high-pressure-side terminal section PP and then to thread it into the pump bed 307 by manually turning the rotor 310 in the direction of the arrows D. As a threading or insertion aid for supporting manual threading, the known peristaltic pump has two guiding projections 380 carried by the rotor 310 in the form of hooks 360, which are arranged between the two pinch rollers 320. In contrast to concepts with automatic threading of the pump segment, in which the rotor is automatically aligned in an optimum threading position, in the case of the peristaltic pump known from document EP 1 749 549 B1 the user has to ensure that the tube is inserted upstream in front of the hook 360 in order to prevent jamming. For this purpose, the rotor 310 has to be manually aligned as shown in FIG. 8. In order to guide the tube segment already gripped by the hooks 360 and to align it centrally to the squeeze element, guiding pins 370, 390 are additionally used which are inserted into the rotor and serve as tube guiding elements, which follow the hooks 360.

### SUMMARY

The present disclosure is based on the object of further developing a generic peristaltic pump in such a way that the threading process is simpler for the user while simplifying the design, wherein the tube segment to be threaded into the peristaltic pump is to be protected as far as possible from damage.

According to the new development, both the insertion aid and the tube guiding elements are integrated into the swing arm adjacent to the squeeze element in the rotational direction of the rotor. This reduces the number of parts and therefore also the manufacturing and assembly costs, since only the assembly of the swing arm is required. Since the insertion aids are adjacent to, i.e. directly in front of, the associated squeeze element, manual insertion of the pump segment is possible without the user having to pay attention to a specific position of the rotor.

Advantageously, the swing arm is attached to the rotor via a pivot bearing, which is arranged offset to the squeeze element in the rotational direction of the rotor, wherein the insertion aid and the tube guiding elements are located on the pivot bearing. With this configuration, the structure of the swing arm with integrated insertion aid and tube guiding can be kept particularly compact.

Tests have shown that, due to the novel assembly of the insertion aid on the pivot bearing of the swing arm, it is sufficient for gentle and reliable threading of the tube segment to form the insertion aid as a simple, pin-like guide or conduit body which extends from the pivot bearing substantially in the radial direction away from the rotor axis in the direction of the support surface and which, viewed in cross-section, has substantially a rounded wedge shape pointing in the rotational direction of the rotor, which is angled toward the pump bed at an acute angle, preferably between 3° and 50°.

It is furthermore advantageous if the conduit body has two functional surfaces, of which the outer functional surface leading in the rotational direction of the rotor and facing away from a central plane of the swing arm forms a rounded wedge surface, and the inner functional surface facing the central plane is formed by a cylinder segment surface that adjoins the wedge surface in a preferably stepless manner. The rounded wedge surface presses the tube segment to be threaded gently downward into the pump bed when the rotor is turned manually, wherein the rounded geometry prevents damage to the tube segment. The cylinder segment surface adjacent to the wedge surface can advantageously be used to guide the tube segment centrally to the squeeze elements during operation of the peristaltic pump. Of course, care is taken to ensure that the quality of the surfaces of the conduit body that come into contact with the tube segment is such that the tube segment is protected as much as possible.

The cross-section of the conduit body may have a wide variety of shapes as long as the wedge surface and the cylinder segment surface on the side facing the tube segment are formed in accordance with the application. However, if the cross-section of the conduit body has a central axis, production is simplified, in particular if the conduit body is attached to the swing arm as a separate component. In the alternative case that the swing arm and the conduit body are configured as one piece, it is also advantageous if the conduit body is additionally supported on the swing arm via a brace or crosspiece following the central axis. This design allows the conduit body to be filigree and yet stable, which even opens up the possibility of forming it with a hollow profile.

If the conduit body is arranged on a pivot bearing eye of the swing arm, preferably attached to it in one piece, the radial support forces and bending forces applied to the conduit body during threading can be easily transferred to the swing arm, since the pivot bearing eye is stably supported by the pivot axis and is therefore only subject to minimal deformation.

A further advantageous design of the peristaltic pump is to equip the swing arm with an abutment or stop surface for a pressure spring at its end portion facing away from the pivot bearing eye and to arrange—for example rotatably mount—the squeeze element between the pivot bearing eye and the stop surface. This gives the swing arm a compact form, leaving sufficient free space around the circumference of the rotor for the formation of further functional surfaces, such as guide surfaces for the tube segment.

Preferably, the first conduit body forming the insertion aid described above is assigned a further, second conduit body, which is configured and oriented symmetrically to the first conduit body with respect to a horizontal plane parallel to the pump bed. Thus, the facing cylinder segment surfaces of the two conduit bodies arranged in mirror image form the functional surfaces of the tube guiding elements. The integration of the insertion aid and the tube guiding elements into the swing arm is ensured with a further reduction in manufacturing effort. Due to the mirror-image arrangement and alignment of the conduit bodies, they guide the tube segment gently and like a funnel into the center of the relevant squeeze element during operation of the peristaltic pump, which has a positive effect on the delivery accuracy of the peristaltic pump.

The manufacture of the swing arm is further simplified if the horizontal plane, to which the two conduit bodies are arranged symmetrically, is at the same time a symmetry plane of the swing arm.

The tube segment is advantageously protected even better against wear by the fact that the insertion aid and preferably also the tube guiding elements are made of and/or coated with a material that has abrasion-minimizing sliding properties.

The swing arm with integrated insertion aid and integrated tube guiding elements according to the application can be manufactured in a wide variety of ways. It may also consist of different materials and may be assembled to form an integral unit, in which case a wide variety of shaping manufacturing processes, such as CNC milling technology, may be used. There are particular advantages in terms of production, assembly and material consumption if the swing arm is configured as a one-piece component manufactured using the metal injection molding (MIM) process. It has been shown that this manufacturing process already ensures a surface quality without reworking that guarantees the greatest possible protection of the tube segment. It should be noted that the manufacture of the swing arm using the MIM (Metal Injection Molding) process is to be regarded as a separate aspect of the present disclosure for which protection is sought specifically, irrespective of the geometric design of the swing arm. In this case, the swing arm of the rotor is preferably made of a high-strength stainless steel with very good chemical properties. This production method enables the integration of additional functions, such as threading aid and tube segment guide, while at the same time significantly reducing the raw material used and the manufacturing costs compared to machining. Mechanical reworking can be kept to a minimum thanks to the shaping MIM process. In addition, filigree contours (e.g. reinforcing ribs) can be realized using the MIM process, whereby the mass of the component can be reduced even further while maintaining the same strength. These filigree contours are difficult or impossible to produce using milling technology. Due to the good surface quality that can be achieved using the MIM process, no special reworking is required in the area of the surfaces that come into contact with the tube segment, which may also be used to reduce production costs. Another powder injection molding process may also be used to produce the swing arm, for example ceramic powder injection molding.

Tests have shown that such materials, such as an austenitic, heat-resistant chromium-nickel steel known as 1.4841 (X15CrNiSi25-21), can easily be produced with sufficiently high densities using the MIM process and that the components made from them have optimum properties for use in peristaltic pumps. The swing arm produced in this way is chemically resistant to a wide range of media and is so strong with only a few cm³ of material used that, on the one hand, the pressure force of the rollers is ensured during conveying operation of a wide variety of media with a delicate design and, on the other hand, the flanging of the roller axles is guaranteed without deforming the swing arms. An additional advantage is that the chemical properties of the material make additional surface treatment such as painting or anodizing unnecessary.

The insertion aid and the tube guiding geometry are integrated into the swing arm. This reduces manufacturing and assembly costs. The tube guiding geometry has two regions. The area marked with a dotted line on the upper horn in the illustration in FIG. 6 is a rounded, angled surface that presses the tube down when threading. The rounded geometry prevents damage to the tube. The round surfaces on the upper and lower horns marked with a dashed line in FIG. 6 position the tube during operation so that the pump segment is guided centrally over the pinch rollers. The quality of the surfaces in the area of the tube guide is selected so that the tube is not damaged. The surface quality can be produced without special reworking. Due to the assembly of the insertion aid directly in front of the pinch rollers, the user does not have to pay attention to any special position of the rotor during manual threading.

### BRIEF DESCRIPTION OF THE DRAWINGS

Configuration examples of the present disclosure are explained in more detail below with reference to schematic drawings. The following is shown:

FIG. 1 shows a perspective representation of an exemplary embodiment of a peristaltic pump as it can be used in connection with a device for extracorporeal blood treatment;

FIG. 2 shows a perspective view of the peristaltic pump according to FIG. 1 with inserted tube segment, which forms the pump segment;

FIG. 3 shows a perspective view of a rotor used in the peristaltic pump according to FIGS. 1 and 2;

FIG. 4 shows a perspective view of the rotor base body of the peristaltic pump with attached swing arm;

FIG. 5 shows a perspective view of the rotor from a different viewing direction to FIG. 2;

FIG. 6 shows a highly magnified side view of the swing arm used in the peristaltic pump according to FIGS. 1 and 2, viewed in the direction of arrow ‘VI’ in FIG. 5;

FIGS. 7A to 7D show schematic top views of the peristaltic pump to represent the different phases of the threading of the pump segment;

FIG. 8 shows a schematic top view of a peristaltic pump according to the state of the art; and

FIG. 9 shows a perspective view of the rotor used in the peristaltic pump according to FIG. 8.

### DETAILED DESCRIPTION

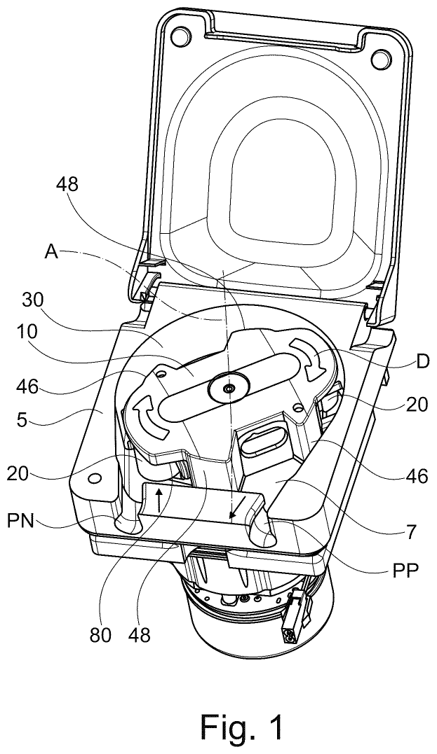

FIG. 1 shows a peristaltic pump as used in dialysis machines, for example. The object of the peristaltic pump is to pump or convey a defined volume of a medium, such as blood or dialysis fluid, by deforming and clamping the elastically deformable fluid line. The peristaltic pump for pumping blood generally pumps from a negative pressure side PN (low-pressure connection or terminal section) to a positive pressure side PP (high-pressure connection or terminal section).

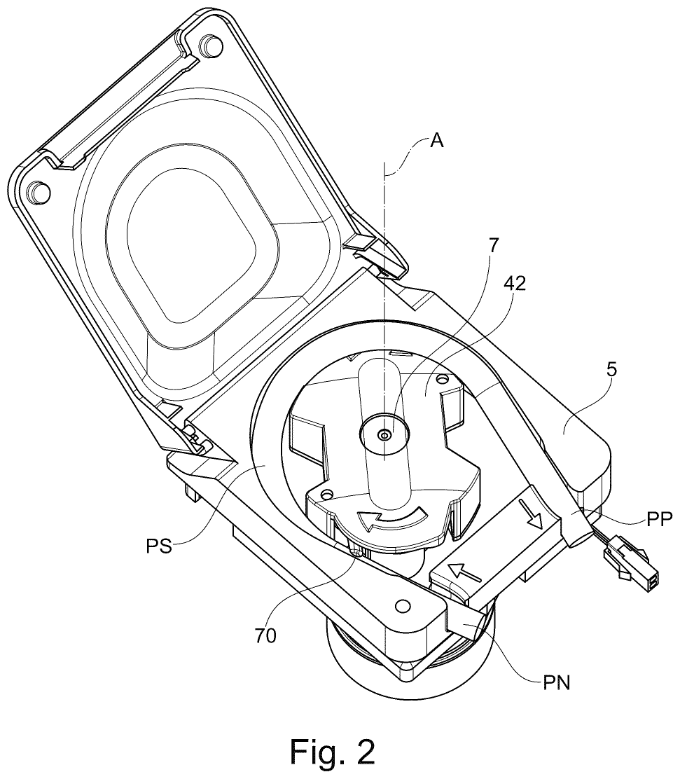

As shown in FIGS. 1 to 3, the peristaltic pump has a rotor or pump housing 5—shown unfolded in FIG. 1—in which a rotor 10 rotatable about a rotor axis A is housed with at least two squeeze elements 20, in this case pressure rollers, which are for example diametrically offset from each other in the circumferential direction. The pump housing 5 comprises a pump bed 7, which extends arc-shaped around the rotor axis A and has a support surface 30 radially spaced from the rotor 10. This support surface 30 is configured to radially support a pump segment PS, shown in FIG. 2, which can be inserted between it and the rotor 10. The squeeze elements 20 are, for example, spring-mounted on a swing arm not shown in FIG. 1, which is in turn pivotably movable and attached to the rotor 10 via a hinge pin 38.

The tube segment used in the peristaltic pump, i.e. housed in the pump bed 7, is referred to below as the pump segment. The tube segment not shown in FIG. 1 is supported radially in the pump bed 7 on a support surface 30. The support surface 30 is usually formed by a cylinder surface, but it may also be recessed in a trough shape. The rotational direction of the rotor 10 when pumping fluid is indicated by the arrow D.

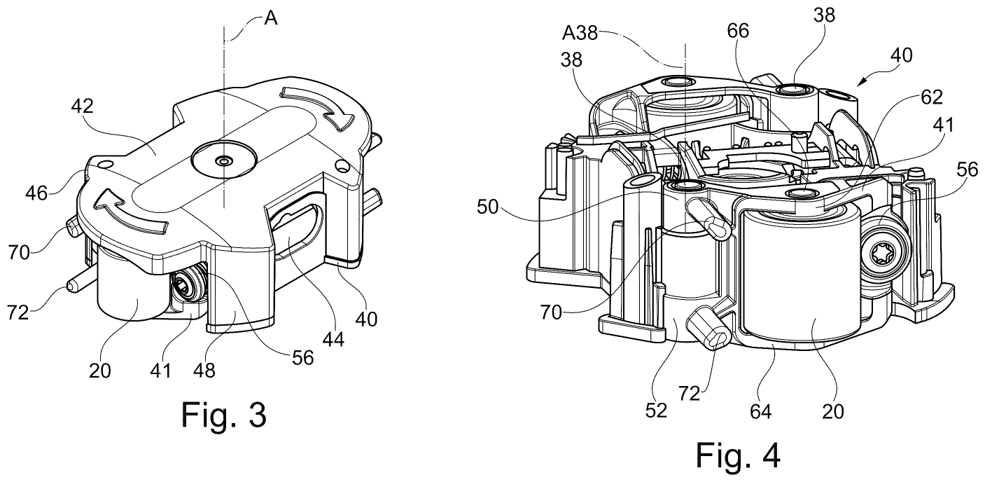

FIG. 3 shows details of the rotor 10. It has a rotor base body 40, which is not shown in detail, and a rotor cover 42. The rotor 10 can be removed from a drive shaft, which is not shown in detail, by a laterally operable button 44. The squeeze elements 20 are rotatably mounted on a swing arm 41 resiliently supported on the rotor base body 40.

The rotor cover 42, which co-rotates with the rotor 10, has guide surfaces 46, 48 in the area of the squeeze elements 20, of which one guide surface 46 runs in front of the squeeze element 20 and the other guide surface 48 runs behind the squeeze element 20. The guide surfaces 46, 48 are therefore arranged and configured adjacent to each other on both sides of the squeeze element 20 in the circumferential direction in such a way that they each form a circular segment-like guiding passage with the support surface 30, in which a pump segment inserted into the pump bed 7 is radially trapped with a predetermined clearance fit. During operation of the peristaltic pump, the pump segment can be fixed to the pump bed 7 via fitting bodies, which are not shown in detail and which can be secured against displacement in corresponding connections or terminals of the pump housing 5.

Before the peristaltic pump is put into operation, a tube segment, which becomes the pump segment of the system when the pump is in operation, has to be inserted or installed into the pump bed 7 and positioned centrally in relation to the squeeze element 20. The initial phase of this threading is shown in FIG. 2.

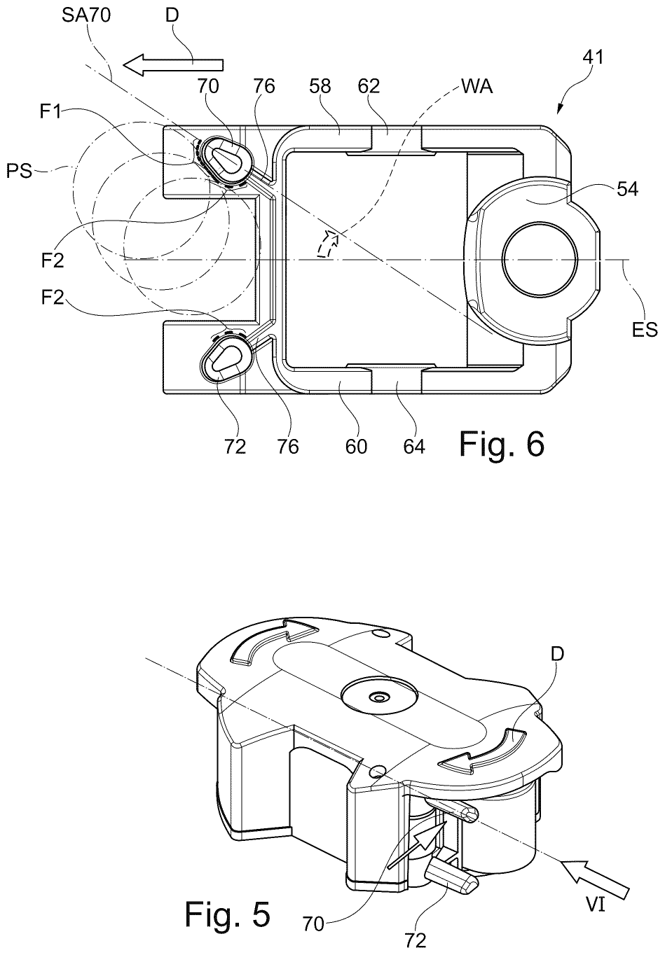

The pump segment designated PS is first placed in the pump bed 7—as shown in FIG. 2—in such a way that it extends from the low-pressure-side terminal section PN arc-shaped above the rotor 10 following the support surface 30 to a high-pressure-side terminal section PP. In this phase, the pump segment PS is still completely above the rotor 10, more precisely above the rotor cover 42. In order to now thread the pump segment PS into the pump bed 7 by manually turning the rotor 10 in the direction of arrow D and to keep the stresses on the pump segment PS as low as possible and the handling as simple as possible, the rotor 10 carries an insertion aid with which the tube or pump segment PS can be pressed into the pump bed 7 during the threading process, as well as tube guiding elements with which the tube or pump segment PS can be aligned centrally to the squeeze elements 20. The insertion aid and the tube guiding elements are described in more detail below with reference to FIGS. 4 to 6.

For this innovation, the swing arm 41, which is pivotably movably attached to the rotor base body 40, is the carrier of the insertion aid and the tube guiding elements. More precisely, the insertion aid and tube guiding elements are integrated into the swing arm 41 as follows:

As shown in FIG. 4, which shows the rotor base body 40 from which the rotor cover 42 is removed, the swing arm 41 is pivotably fixed to the hinge pin 38 with the pivot axis A38 via two bearing eyes 50, 52. As can be seen from FIGS. 4 and 6, the swing arm 41 has, at its end portion facing away from the bearing eyes 50, 52, a stop surface 54 for a pressure spring 56 with which the swing arm 41 is subjected to a compressive force directed away from the rotor base body 40. Between the bearing eyes 50, 52 and the stop surface 54, the swing arm 41, as can be seen from the representation according to FIG. 4, is formed in the form of a slightly convexly curved bracket with two bracket arms 58, 60 lying at a parallel distance, which in turn form bearing sections 62, 64 for a rotational axis 66 (see FIG. 4) of the pinch roller 20. The swing arm 41 has a symmetry plane ES, to which the pinch roller 20 is also positioned centrally in this way.

Guide or conduit bodies 70, 72, which are integrated into the swing arm 41 as close as possible to the pinch rollers 20, serve as insertion aids and as guide elements for the pump segment PS on the pivot bearing, or more precisely on the bearing eyes 50, 52, wherein the conduit bodies 70, 72 extend so far away from the pivot bearing, i.e. from the axis A38 of the hinge pin 38, substantially in the radial direction away from the rotor axis A, i.e. from the rotor base body 40, that they come as close as possible to the support surface 30 when the rotor 10 rotates—as can be seen in FIG. 2, in which, however, only an upper conduit body 70 is visible.

The upper conduit body 70 forms the insertion aid, the function of which is described in more detail below, and both conduit bodies 70 and 72 together act as tube guiding elements, with which the pump segment PS is aligned centrally to the squeeze elements or pinch rollers 20.

At least the upper conduit body 70—in the configuration example shown both conduit bodies 70 and 72—forming the insertion aid substantially have a rounded wedge shape in cross-section and in the radial view according to FIG. 6, pointing in the rotational direction D of the rotor, which is angled at an acute angle WA (see FIG. 6) to the pump bed 7 and thus to the symmetry plane ES of the swing arm 41 parallel thereto.

At least the upper conduit body 70 has two functional surfaces F1 and F2, of which the outer functional surface F1 (marked with a dotted line in FIG. 6) leading in rotational direction D and facing away from the symmetry plane ES forms a rounded wedge surface and the inner functional surface F2 (marked with a dashed line in FIG. 6) facing the symmetry plane ES is formed by a cylinder segment surface that preferably adjoins the wedge surface in a continuous manner. The lower conduit body 72 forms at least the inner functional surface F2. The functional surface F1 acts as an insertion aid when threading the pump segment 40, and the opposing functional surfaces F2 act as tube guiding elements in order to keep the pump segment PS centered in relation to the squeeze elements, i.e. the pinch rollers 20, during operation of the peristaltic pump.

In the configuration example shown, the cross-section of the conduit bodies 70, 72 each has a central axis or axis of symmetry, wherein only the axis of symmetry SA70 of the conduit body 70 is shown in FIG. 6. FIG. 6 also shows that the conduit bodies 70, 72 are additionally supported on the swing arm 41 via a brace or crosspiece 76 following the axis of symmetry SA70.

In summary, the upper conduit body 70 with the outer functional surface F1 forms the insertion aid for the pump segment PS, while both conduit bodies 70, 72 together with their functional surfaces F2 serve as tube guiding elements.

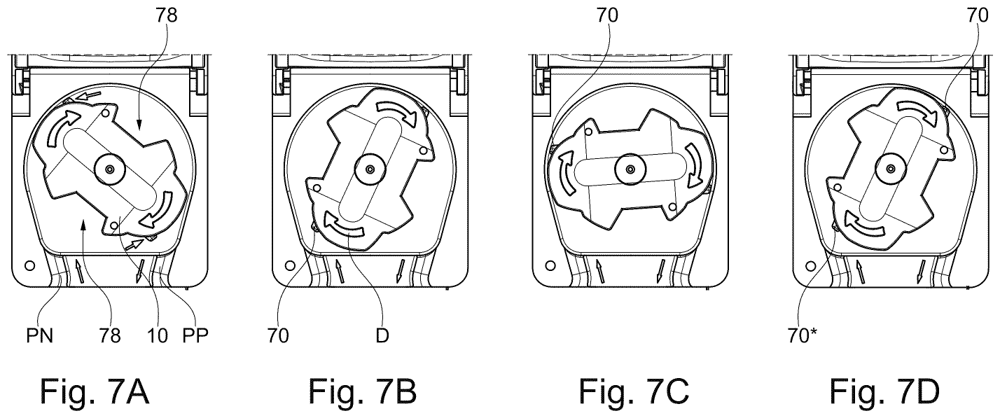

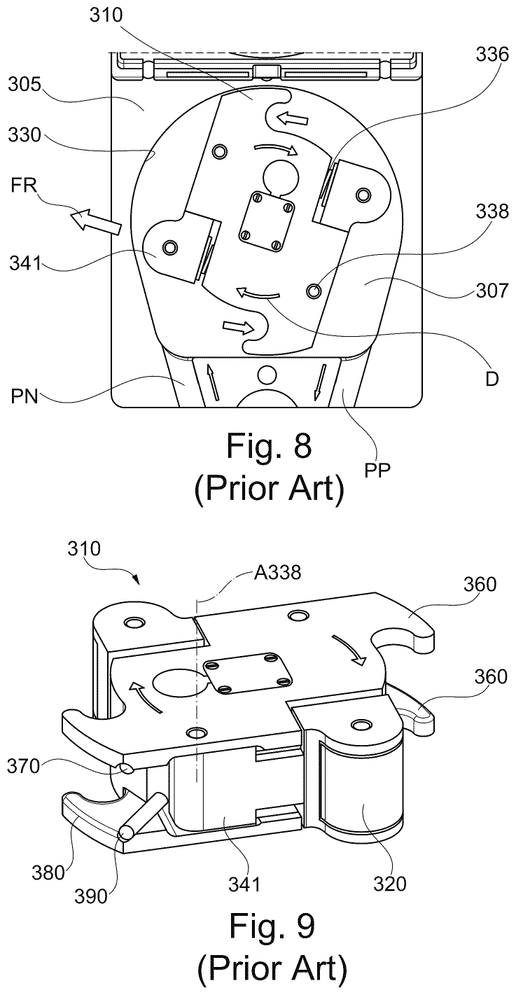

This design results in the following mode of operation, which is described with reference to FIGS. 2 and 7A to 7D:

Starting from any rotational position, for example from the position of the rotor 10 shown in FIG. 7A, above which the inserted pump segment PS extends from the terminal section PN to the terminal section PP following the support surface 30, the rotor 10 can be rotated manually by the user grasping the rotor 10 in the clearance 78 between the guide surfaces 46, 48. The conduit body 70 does not yet touch the pump segment PS in this phase.

As soon as the rotor reaches the rotational pose shown in FIG. 7B, which is also shown in FIG. 2, the outer functional surface F1 of the conduit body 70 slides onto the pump segment PS. This moment is indicated in FIG. 6 by the dotted line representation of the pump segment. As the rotor 10 continues to rotate, the functional surface F1 of the conduit body 70 pushes the pump segment PS further and further down into the pump bed so that the pump segment PS, because it is in contact with the support surface 30, is finally caught by the functional surfaces F2 in the center of the symmetry plane ES. This function continues in the same way when passing over the support surface 30 (see FIG. 7C), so that finally the entire pump segment PS is centered in relation to the pinch rollers in the pump bed 7.

FIG. 7D shows the rotational position of the rotor 10 when the conduit body 70*, which is offset by 180° to the conduit body 70 and was not involved in the threading process, meets the pump segment PS, which is already in the pump bed 7. In this phase, the conduit bodies 70 and 72 approach the pump segment PS in such a way that they guide the pump segment PS between the functional surfaces F2 in a funnel-like manner and thus align it centrally to the pinch rollers 20. This mode of operation occurs every time a pinch roller is activated during operation of the peristaltic pump, which means that the conveyed volume of the peristaltic pump can be kept constant.

Preferably, the conduit bodies 70, 72 are formed and/or coated from a material that has abrasion-minimizing sliding properties.

In the configuration example shown, the conduit bodies 70, 72 are formed integrally with or attached in one piece to the swing arm 41, namely to a pivot bearing eye 50, 52. In this case, an injection molding process can be used to manufacture the swing arm 41, for example a metal injection molding (MIM) process, whereby it is even possible to manufacture the swing arm 41 from a stainless steel material, for example a heat-resistant austenitic chromium-nickel steel (X15CrNiSi25-21), as is known under the designation 1.4841. This steel is particularly resistant to oxidation and it has been shown that the surface qualities achievable in the MIM process are sufficient to make the functional surfaces F1 and F2 smooth enough without further finishing to press the pump segment PS gently and without abrasion into the pump bed and to align it centrally to the pinch rollers.

Of course, deviations from the configuration example described are possible without departing from the basic idea of the present disclosure.

It is also possible, for example, to configure the conduit bodies 70, 72 as separate components, which are then connected to the swing arm 41 to form an integral unit. The conduit bodies 70, 72 do not have to be axially symmetrical to each other. The lower conduit body 70 may, for example, have a circular cylindrical cross-section.

The swing arm 41 with integrated insertion aid and tube guiding elements may also be manufactured from a wide variety of materials and/or using a milling process.

In the peristaltic pump described above, two squeeze elements are provided which are rigidly arranged in a relative position of 180° or at a fixed angle of 180° to each other. However, it is also possible that more than two squeeze elements are provided and that the squeeze elements can be angularly adjustably positioned relative to each other in the direction of rotation. For this purpose, the squeeze elements may be arranged on rotor arms, which may be configured to pivot in a circumferential direction in relation to the rotor.

The squeeze elements do not have to be configured as squeeze rollers or pinch rollers, which advantageously roll along the fluid line in a way that is gentle on the material. Sliding shoes that glide over the tube segment may also be provided.

The present disclosure thus creates a peristaltic pump, in particular for conveying fluid in a device for extracorporeal blood treatment, with a pump housing, in the pump bed of which a rotor rotatable about a rotor axis is housed with at least two squeeze elements offset in circumferential direction to each other and which has a support surface extending arc-shaped about the rotor axis and spaced radially from the rotor, wherein the support surface is arranged to support a tube segment, i.e. the pump segment, which can be inserted between it and the rotor. In order to hold the squeeze element, the rotor has a swing arm, which is pivotably movable and spring-mounted on the rotor, an insertion aid, with which the tube segment can be pressed into the pump bed during the threading process, and tube guiding elements, with which the tube segment can be aligned centrally to the squeeze element.

In order to keep the threading process as simple as possible for the user while simplifying the set-up and to protect the tube segment to be threaded into the peristaltic pump from damage as far as possible, both the insertion aid and the tube guiding elements are integrated into the swing arm adjacent to the squeeze element in the rotational direction of the rotor.

### LIST OF REFERENCE SIGNS

A rotor axis
D rotational direction
PN low-pressure terminal section
PP high-pressure terminal section
PS pump segment
ES symmetry plane
F1 functional surface
F2 functional surface
FR radial direction
5 pump housing
7 pump bed
10 rotor
20 squeeze element
30 support surface
38 pivot bearing/hinge pin
40 rotor base body
42 rotor cover
44 button
46 guide surface
48 guide surface
50 bearing eye
52 bearing eye
54 stop surface
56 pressure spring
58 upper bracket arm
60 lower bracket arm
62 upper bearing section
64 lower bearing section
66 rotational axis
70 upper conduit body
72 lower conduit body
76 crosspiece
78 clearance
305 housing
307 pump bed
310 rotor
320 squeeze elements
330 support surface
341 swing arm
336 pressure spring
338 hinge pin
A338 axis
341 swing arm
360 hook
370 guiding pin
380 guiding projections
390 guiding pin

1. A peristaltic pump for conveying fluid in a device for extracorporeal blood treatment, the peristaltic pump comprising:
a pump housing comprising a pump bed, wherein the pump bed houses a rotor rotatable about a rotor axis, the pump housing further comprising a squeeze element and a support surface extending arc-shaped about the rotor axis and spaced radially from the rotor, wherein the support surface is arranged to support a tube segment which is insertable between the support surface and the rotor,
wherein the rotor comprises a swing arm that is pivotably movable and spring-mounted on the rotor, the rotor also comprising an insertion aid for the squeeze element with which the tube segment is pressable into the pump bed during a manual threading process, the rotor further comprising tube guiding elements, with which the tube segment is centrally alignable to the squeeze element, and
wherein both the insertion aid and the tube guiding elements are integrated into the swing arm adjacent to the squeeze element in a rotational direction of the rotor.

2. The peristaltic pump according to claim 1, wherein the swing arm is attached to the rotor via a pivot bearing, which is arranged offset to the squeeze element in the rotational direction, wherein the insertion aid and the tube guiding elements are located on the pivot bearing.

3. The peristaltic pump according to claim 2, wherein the insertion aid is formed by a conduit body that extends from the pivot bearing substantially in a radial direction away from the rotor axis and which, viewed in cross-section, has substantially a rounded wedge shape pointing in the rotational direction, which is angled toward the pump bed at an acute angle.

4. The peristaltic pump according to claim 3, wherein the conduit body comprises an outer functional surface leading in the rotational direction of the rotor and facing away from a central plane, the outer functional surface forming a wedge surface that is rounded, the conduit body further comprising an inner functional surface facing the central plane, the inner functional surface being formed by a cylinder segment surface that adjoins the wedge surface.

5. The peristaltic pump according to claim 3, wherein the cross-section of the conduit body has a central axis.

6. The peristaltic pump according to claim 5, wherein the conduit body is additionally supported on the swing arm via a crosspiece following the central axis.

7. The peristaltic pump according to claim 3, wherein the conduit body is integrally attached to a pivot bearing eye of the swing arm.

8. The peristaltic pump according to claim 7, wherein the swing arm forms a stop surface for a pressure spring at its end portion facing away from the pivot bearing eye, and wherein the squeeze element is arranged between the pivot bearing eye and the stop surface.

9. The peristaltic pump according to claim 8, wherein the squeeze element is rotatably mounted.

10. The peristaltic pump according to claim 3, wherein the conduit body is assigned a second conduit body configured and oriented symmetrically to the conduit body with respect to a horizontal plane parallel to the pump bed, so that facing cylinder segment surfaces of the conduit body and the second conduit body form functional surfaces of the tube guiding elements.

11. The peristaltic pump according to claim 10, wherein the horizontal plane is a symmetry plane of the swing arm.

12. The peristaltic pump according to claim 1, wherein at least the insertion aid and/or the tube guiding elements are made of and/or coated with a material which has abrasion-minimizing sliding properties.

13. The peristaltic pump according to claim 1, wherein the swing arm with integrated insertion aid and tube guiding elements is configured as a one-piece component.

14. The peristaltic pump according to claim 13, wherein the swing arm is a metal injection molded part.
